(19) United States
(12) Patent Application Publication
(10) Pub. No.: US 2025/0049985 A1
(43) Pub. Date: Feb. 13, 2025

(54) BONE ADHESIVE COMPOSITION

(71) Applicants: INSERM (INSTITUT NATIONAL DE LA SANTÉ ET DE LA RECHERCHE MÉDICALE), PARIS (FR); CENTRE HOSPITALIER UNIVERSITAIRE DE LILLE, LILLE (FR); UNIVERSITE DE LILLE, LILLE (FR); INSTITUT NATIONAL DE RECHERCHE POUR L'AGRICULTURE, L'ALIMENTATION ET L'ENVIRONNEMENT, PARIS (FR); CENTRE NATIONAL DE LA RECHERCHE SCIENTIFIQUE, PARIS (FR); CENTRALE LILLE INSTITUT, VILLENEUVE D'ASCQ (FR)

(72) Inventors: Mathias SCHLUND, LILLE (FR); Feng CHAI, LOOS (FR); Joël LYSKAWA, COUTICHES (FR); Joël FERRI, VILLENEUVE D'ASCQ (FR)

(73) Assignees: INSERM (INSTITUT NATIONAL DE LA SANTÉ ET DE LA RECHERCHE MÉDICALE), PARIS (FR); CENTRE HOSPITALIER UNIVERSITAIRE DE LILLE, LILLE (FR); UNIVERSITE DE LILLE, LILLE (FR); INSTITUT NATIONAL DE RECHERCHE POUR L'AGRICULTURE, L'ALIMENTATION ET L'ENVIRONNEMENT, PARIS (FR); CENTRE NATIONAL DE LA RECHERCHE SCIENTIFIQUE, PARIS (FR); CENTRALE LILLE INSTITUT, VILLENEUVE D'ASCQ (FR)

(21) Appl. No.: 18/720,043

(22) PCT Filed: Dec. 16, 2022
(86) PCT No.: PCT/FR2022/052402

§ 371 Date: Jun. 14, 2024

(30) Foreign Application Priority Data
Dec. 17, 2021 (FR) FR2113780

(51) Int. Cl. A61L 24/00

(52) U.S. Cl. CPC A61L 24/0063

(57) ABSTRACT

An adhesive composition including a calcium phosphate ceramic selected from tetracalcium phosphate and alpha-tricalcium phosphate, phosphorylated serine, polydopamine, and an aqueous solvent. Also, a kit for preparing the adhesive composition, which includes the calcium phosphate ceramic, phosphorylated serine, and polydopamine and a method for preparing the adhesive composition, as well as the uses thereof.

### FIELD

The present invention relates to an adhesive composition comprising a calcium phosphate ceramic selected from tetracalcium phosphate and alpha-tricalcium phosphate, phosphorylated serine, and polydopamine, the preparation method thereof and the use thereof.

### BACKGROUND

Bone fracture is a very common pathology. The treatment of fractures is based on the reduction of the fracture site then the restraint thereof until consolidation. The restraint can be obtained either orthopedically by maxillo-mandibular blockage at the facial level or by plaster at the limb level, or surgically by osteosynthesis. The surgical treatment, allowing a faster return to function, is today widely preferred to orthopaedic treatment with the exception of particular specific fractures and paediatric cases.

The surgical restraint currently relies on metal osteosynthesis material, mainly consisting of metal plates which are screw-retained on either side of the fracture line. Moreover, the fractures are not the only interventions where a bone fixation is necessary. The bone autograft techniques for filling losses of bone substances or for preprosthetic purposes at the craniofacial level require the fixation of the bone fragments to prevent any mobility of the graft. The metal material is also used for the bone fixation in surgical osteotomies (orthognathic surgery, tibial osteotomy) and spinal fusions. Finally, the integration of metal implants into the bone (dental implants, joint implants) requires an osseointegration time which could be reduced by the addition of an adhesive. In the cases where a metal implant would be necessary, the adhesive could ensure the fixation thereof.

The cases of comminuted (multi-fragmentary) fractures are particularly complex to be treated with metal material due to the small size of the fragments relative to the size of the screws. The middle and upper third of the face offers a good example of these fractures which are difficult to be treated with the conventional screw-retained plate model due to generally very comminuted fractures in a thin and fragile bone. The limbs are also subject to this type of fracture in certain high-velocity traumas. Moreover, the intra-articular fractures pose a problem since the material can interfere with the functioning of the joint. Finally, because the metal material does not follow the growth of the bones, it is poorly adapted to the cases of paediatric fractures in a growing skeleton.

A bone adhesive or “bone glue” would offer a simple and rapid solution for the management of the fractures, in particular the fractures for which the current metal implant systems are poorly adapted. Bioresorbable, it would allow avoiding the infectious and mechanical complications (undoing of the material) related to metal implants and the numerous interventions carried out to remove this material, interventions related to a risk of post-operative morbidity and to a certain economic burden (hospitalisation and additional surgical procedure, work stoppage).

There is currently no marketed alternative to metal osteosynthesis material, such as a bone adhesive. The main pitfall being the adhesion in a humid physiological environment. Studies and patents have already been published combining the calcium phosphate ceramic (CPC) and phosphorylated serine (O-Phospho-Serine/OPS). The calcium phosphate ceramics have an excellent biocompatibility and osteoconductive properties. They have been used in routine clinical practice for many years as a bone substitute. However, the main problem with calcium phosphate ceramics for bone fixation is their lack of adhesion to bone tissue.

Inspired by the adhesion mechanisms observed in nature, in particular marine animals, phosphorylated serine can be added to a calcium phosphate ceramic in order to obtain adhesive properties. Phosphorylated serine is a molecule used by the marine worm Phragmatopoma californica (or Sandcastle worm) which has the ability to build underwater protective shells by gluing minerals using a complex coacervation mechanism. In addition, the structure of phosphorylated serine is close to osteopontin, which gives it an osteoinductive character.

Bone adhesives consisting of phosphorylated serine and calcium phosphate ceramic have already been studied with a confirmation of the ex vivo adhesive effectiveness and the in vivo biocompatibility, but no study has confirmed the adhesive capacity of this composition in vivo (Bioinspired Mineral—Organic Bioresorbable Bone Adhesive, Kirillova et al., Advanced Healthcare Materials, 2018, doi: 10.1002/adhm.201800467).

It is therefore necessary to be able to have an adhesive composition which is prepared from calcium phosphate ceramic and phosphorylated serine having an adhesive capacity in vivo while being biocompatible and safe.

Continuing this research with a large number of works, the applicants found that an adhesive composition comprising tetracalcium phosphate, phosphorylated serine and polydopamine had such characteristics.

Other features and advantages of the present invention will appear on reading the following detailed description.

### SUMMARY

A first object of the present invention relates to an adhesive composition comprising:
a calcium phosphate ceramic selected from tetracalcium phosphate and alpha-tricalcium phosphate,
phosphorylated serine,
polydopamine, and
an aqueous solvent.

A second object of the present invention relates to a kit for preparing an adhesive composition according to the invention, comprising:
a calcium phosphate ceramic selected from tetracalcium phosphate and alpha-tricalcium phosphate, preferably tetracalcium phosphate,
phosphorylated serine, and
polydopamine.

A third object of the present invention relates to a method for preparing an adhesive composition according to the invention comprising the following steps:
a) Mixing a calcium phosphate ceramic selected from tetracalcium phosphate and alpha-tricalcium phosphate, phosphorylated serine and polydopamine in a container,
b) Adding a solvent to the previous preparation,
c) Recovering the mixture thus formed.

A last object of the present invention relates to the composition according to the invention for its therapeutic use in vivo as a bone adhesive.

### BRIEF DESCRIPTION OF THE DRAWINGS

FIG. 1 Results of the mechanical tensile tests on titanium. Average maximum stress (MPa) of the glued samples (n=8). * means significant difference (p=0.0029).

FIG. 2 Results of the mechanical tensile tests on bovine bones. Average maximum stress (MPa) of the glued samples (n=8). * means significant difference (p=0.00093).

FIG. 3 Results of the ex vivo tensile tests of the adhesive compositions according to Example 1. Average maximum stress (MPa) of the glued samples (n=7). * means significant difference (p=0.029).

### DETAILED DESCRIPTION

### The Adhesive Composition

The adhesive composition according to the invention comprises:
a calcium phosphate ceramic selected from tetracalcium phosphate and alpha-tricalcium phosphate, preferably tetracalcium phosphate,
phosphorylated serine,
polydopamine, and
an aqueous solvent.

The calcium phosphate ceramic is composed of a biocompatible, polyvalent metal salt that reacts with the phosphorylated serine, an organic phosphate compound, in an aqueous environment to form compositions having powerful adhesive properties.

The calcium phosphate ceramic according to the present invention is selected from tetracalcium phosphate, for example that from Matexcel or Hangzhou ICH Biofarm, and alpha-tricalcium phosphate, for example that from Matexcel, Innotere or Merck. Preferably, the calcium phosphate ceramic is tetracalcium phosphate.

The amount of calcium phosphate ceramic in the composition can vary and is preferably comprised between 50% and 80%, preferably between 60% and 70%, more preferably between 65 and 68% by dry weight on the total weight of the composition.

The phosphorylated serine according to the present invention, for example O-Phospho-DL-Serine or O-Phospho-L-Serine from Merck, is a biocompatible molecule used by the marine worm Phragmatopoma californica (or Sandcastle worm) which has the ability to build underwater protective shells by gluing minerals using a complex coacervation mechanism. In addition, the structure of phosphorylated serine is close to osteopontin, which gives it an osteoinductive character.

The amount of phosphorylated serine in the composition can vary and is preferably comprised between 20% and 50%, preferably between 30% and 40%, more preferably between 32% and 35% by dry weight on the total weight of the composition.

Preferably the calcium phosphate ceramic/phosphorylated serine dry weight ratio is comprised between 1.5 and 2.5, preferably between 1.7 and 2.3, more preferably between 1.9 and 2.

Polydopamine, particularly in nanoparticle form (nPDA), is biocompatible and has excellent stimulatory properties for cell adhesion, cell proliferation and osteogenic differentiation. The porous and hydrophobic structure thereof allows the formation of covalent and hydrogen bonds with the hydroxyapatite which is the main component of bone tissue. In addition, polydopamine induces an apatitic mineralisation by providing nuclear sites for the calcium phosphate mineralisation in Simulated Body Fluid (or SBF).

The term “nanoparticle” means a particle size comprised between 100 nm and 400 nm, preferably between 125 and 275 nm.

Nanoparticulate polydopamine can be synthesised according to the protocol of Ju et al. Bioinspired Polymerization of Dopamine to Generate Melanin-Like Nanoparticles Having an Excellent Free-Radical-Scavenging Property. Biomacromolecules. 2011-3-14; 12 (3): 625-32.

The amount of polydopamine in the composition can vary and is preferably comprised between 1% and 5%, preferably 2% by dry weight on the total weight of the composition.

According to one aspect, the calcium phosphate ceramic, phosphorylated serine and polydopamine are in powder form.

According to a particular embodiment, wherein the polydopamine is functionalised with at least one active ingredient selected from antibiotics, osteoinductive molecules or osteoinductive peptides, and x-ray contrast agents. Indeed, its particulate, and preferably nanoparticulate, form, its hydrophobic nature and its reactivity with regard to nucleophiles, allows containing active ingredients selected from antibiotics and osteoinductive molecules or osteoinductive peptides, and x-ray contrast agents. The antibiotics would allow preventing and/or treating the bacterial infections which are a major complication causing a very high morbidity in the fractures. Examples of antibiotics are ciprofloxacin, gentamicin, vancomycin, tobramycin, and rifampicin, preferably ciprofloxacin. The osteoinductive molecules would allow accelerating the ossification and thus the bearing. Examples of osteoinductive molecules are simvastatin, anti-BMP2 (Bone Morphogenetic Protein 2) antibodies. Examples of osteoinductive peptides are Osteogenic growth peptide, PepGen P-15 cell binding peptide, the peptides containing the tripeptide motif Arg-Gly-Asp (RGD) “RGD containing peptide”, the synthetic collagen-mimetic peptide of GFOGER sequence or DGEA sequence, or even the BMP-2 protein. Examples of x-ray contrast agents are metals such as Cu²⁺, Mn²⁺, Fe³⁺, or even Gd²⁺. Said metals are chelated by polydopamine and are particularly adapted for MRI.

In this manner, the composition according to the invention has both an adhesive capacity in vivo, a biocompatibility, but also an ability to potentiate bone ossification or to prevent and/or treat the undesirable effects of a bone fracture.

The functionalisation of polydopamine, and in particular of polydopamine nanoparticles, can be done by simple hydrophobic/hydrophobic interactions or by ionic interactions (non-covalent bonds), or by a reaction called Michael reaction or Schiff base formation via the amine or thiol functions of the peptides or proteins (covalent bonds). Examples of functionalisation of polydopamine nanoparticles with antibiotics are described in Yu Fu et al. Mater. Horiz., 2021, 8, 1618-1633. Examples of functionalisation of polydopamine nanoparticles with osteoinductive molecules are described in Ko et al., Biomacromolecules, 2013, 14, 3202-3213. Examples of functionalisation of polydopamine nanoparticles with Cu²⁺ are described in Rui Ge et al. “Cu2+-Loaded Polydopamine Nanoparticles for Magnetic Resonance Imaging-Guided pH- and Near-Infrared-Light-Stimulated Thermochemotherapy” ACS Applied Materials & Interfaces 2017 9 (23), 19706-19716. Examples of functionalisation of polydopamine nanoparticles with Fe³⁺ are described in Qu J et al. “Synthesis of Biomimetic Melanin-Like Multifunctional Nanoparticles for pH Responsive Magnetic Resonance Imaging and Photothermal Therapy”. Nanomaterials (Basel). 2021-8-19; 11 (8): 2107. Examples of functionalisation of polydopamine nanoparticles with Gd²⁺ are described in Wang Z et al., “High Relaxivity Gadolinium-Polydopamine Nanoparticles”. Small. 2017 November; 13 (43). Examples of functionalisation of polydopamine nanoparticles with Mn²⁺ are described in Dong Z et al., “Polydopamine Nanoparticles as a Versatile Molecular Loading Platform to Enable Imaging-guided Cancer Combination Therapy”. Theranostics 2016; 6 (7): 1031-1042.

In one embodiment, the calcium phosphorus ceramic, phosphorylated serine, and polydopamine react together to form an adhesive composition when they are combined with an aqueous solvent. The composition can therefore further comprise an aqueous solvent. The aqueous solvent can be water, in particular demineralised water, or a saline solution, in particular a phosphate buffered saline, or a 0.9% NaCl solution. Preferably the aqueous solvent is a phosphate buffered saline.

The amount of aqueous solvent can vary and is preferably comprised according to a solvent/dry composition ratio comprised between 0.19 mL/g and 0.22 mL/g, and preferably, according to a ratio of approximately 0.21 mL/g. This solvent/dry composition ratio allows obtaining a density comprised between 2 g/cm³ and 2.2 g/cm³. In another embodiment, the adhesive composition according to the invention may further comprise an additive. This additive can be used to impart an additional functionality to the composition according to the invention, such as improving or affecting the handling, texture, durability, strength or resorption rate of the material, or to provide additional mechanical, cosmetic or medical properties. For example, polymers or fibres such as poly (lactic-co-glycolic acid) (PLGA)) can be added to improve the mechanical properties of the adhesive composition.

In one embodiment, the adhesive composition allows promoting a new bone growth at the application site, for example by increasing or by stimulating the bone resorption, the deposition or remodelling rate.

### Method for Preparing the Adhesive Composition

A second object of the present invention relates to a method for preparing an adhesive composition according to the invention comprising the following steps:
a) Mixing a calcium phosphate ceramic selected from tetracalcium phosphate and alpha-tricalcium phosphate, preferably tetracalcium phosphate, phosphorylated serine and polydopamine in a container,
b) Adding a solvent to the previous preparation,
c) Recovering the mixture thus formed.

Preferably, the calcium phosphate ceramic, the phosphorylated serine and the polydopamine are in powder form. According to another aspect, polydopamine is in the form of nanoparticle powder.

According to one aspect, the density of the obtained composition is comprised between 2 g/cm3 and 2.2 g/cm3, this density is obtained in particular by respecting a liquid/powder ratio comprised between 0.19 mL/g and 0.22 mL/g, and preferably a ratio of about 0.21 mL/g.

The mixture thus formed can be in the form of a fluid or semi-solid such as a paste, preferably in the form of a paste. This paste can then be used directly by the qualified practitioner on the human or animal subject.

Typically, an obtained adhesive composition has an initial setting or hardening time comprised between 2 and 3 minutes, and a final setting or hardening time comprised between 4 and 8 minutes. This setting time advantageously allows the qualified practitioner to be able to use it during surgery and to be able to wait for the final setting before closing the subject.

In all embodiments, a qualified practitioner, for example, a physician, a dentist, a surgeon, a nurse, or another appropriate person can modify the specific components to obtain the desired adhesive properties of said composition depending on the intended use or the desired result.

### Use of the Adhesive Composition

The adhesive composition can be used in a wide variety of applications. An object of the present invention relates to the composition according to the invention for its therapeutic use in humans or animals, preferably for the use thereof in medical procedures in particular during bone tissue surgery, and more preferably, for the use thereof in vivo as a bone adhesive. The bone tissue surgery can be dental, sinus, facial or other skeletal region surgery.

For example, the adhesive composition can be used to adhere a structure to a surface.

The term “structure” means a solid object. The structure may be a bone or other bone or bone fragment, an implant, a graft, a device, or biological tissue. Examples of biological tissues may be a tendon or a ligament, typically the anterior or posterior cruciate ligament.

The term “surface” means a biological surface. This biological surface can be that of a bone and in particular, the periosteum, a thin connective envelope enveloping the bone only on surfaces which are not covered by cartilage, or the compact bone, a peripheral and dense portion of the bone, or even the spongy bone, a central portion of the bone, or that of a tendon or a ligament.

In one embodiment, the surface is prepared in order to receive the adhesive composition, for example by sharpening the surface of the bone.

In one embodiment, the adhesion of the structure to the surface through the application of the adhesive composition is permanent or intended to be permanent or until the adhesive composition is resorbed or replaced by bone.

In one embodiment, the adhesive composition is used to fill a space, a hole or a void in said surface, either before or after the placement of said structure.

In some embodiments, the adhesive compositions are used to repair a defect in a bone caused by a disease or condition, such as cancer (for example, osteosarcoma), osteoporosis, rickets, a malignant bone tumour, a bone infection, or another genetic or developmental disease.

In some embodiments, the adhesive compositions are used to strengthen a bone in a subject who has been weakened by a disease or condition, such as cancer (for example, osteosarcoma), osteoporosis, rickets, a malignant bone tumour, a bone infection, or another genetic or developmental disease.

In some embodiments, the subject has suffered trauma, such as a broken bone, a fractured bone, a ruptured ligament, a ruptured tendon, or a damaged tooth. Typically, the adhesive composition can allow repairing the ruptured ligament or the ruptured tendon, for example during a rupture of the cruciate ligament to once again ensure mobility of the knee joint.

In some embodiments, the subject undergoes a plastic or reconstructive surgery procedure.

The compositions and the methods can be used to treat a subject suffering from or afflicted with any disease or condition which has an impact on the structural integrity of the bony skeleton or the fibrous connective tissue.

According to another aspect, the adhesive composition according to the invention can be used ex vivo.

### Kit

An object of the present invention relates to a kit for preparing an adhesive composition according to the invention, comprising:
a calcium phosphate ceramic selected from tetracalcium phosphate and alpha-tricalcium phosphate, preferably tetracalcium phosphate,
phosphorylated serine, and
polydopamine.

Typically each component is in powder form, preferably the polydopamine is in the form of nanoparticle powder.

Each component may be packaged in separate form, or one component may be separated in the container thereof and the other two components can be mixed in the same container, or all the components are packaged together in the same container.

In one embodiment, the kit further comprises an aqueous solvent. In this case, the aqueous solvent can be in a separate container and the other components, in particular in powder form, can be mixed in the same container or separated each in different containers or one component is separated in one container and the other two components are mixed in a different container. Preferably, containers which are used are sealed in accordance with good packaging practices to preserve the shelf life of the mixed or separated components. In some embodiments, preserving the shelf life of the components in the kit includes maintaining sterility. If additives are included in said kit, they may be mixed with one or all components or present in a separate container.

Said kit may comprise additional components for the preparation or application of the adhesive composition according to the invention, such as bowls or mixing surfaces, stirring sticks, syringes, catheters adapted to the syringes, for example catheters having a large gauge size, typically 10G, spatulas, syringes, UV or infrared heat guns or other preparation or distribution devices.

### EXAMPLES

### Example 1: Preparation of the Adhesive Composition According to the Invention

Nanoparticulate Polydopamine (nPDA) Synthesis

900 mg of dopamine hydrochloride was dissolved in 450 mL of pure water. The solution was heated to 50° C. and 3.8 mL of sodium hydroxide was added. The solution was maintained at 50° C. for 5 h. The solution was then dialysed using Spectra/Por 6 membranes (Spectrum Labs, Repligen, Waltham, MA, United States) in order to obtain nPDA in suspension. The powder nPDAs were obtained after lyophilisation of this solution.

### Preparation of the Composition

183 mg of tetracalcium phosphate (TTCP) powder from Matexcel, with 92.5 mg of O-Phospho-DL-Serine powder from Merck (OPS) and 5.5 mg of nanoparticulate polydopamine (nPDA) powder, as obtained above, were poured into a mortar where they were mixed with a spatula then with a pestle in order to obtain a homogeneous mixture. 59 μL of phosphate buffered saline (PBS, pH 7.4 from Merck) were added to the micropipette at a liquid/powder ratio of 0.21 mL/g. The powders and the phosphate buffered saline were mixed for 10 seconds using a spatula to obtain a paste. The obtained adhesive composition is the composition according to the invention.

Without wishing to be bound by any theory, tetracalcium phosphate forms ionic interactions with phosphorylated serine, which, when combined in certain ratios and with polydopamine, react to provide a material having advantageous adhesive capabilities relative to a composition containing only tetracalcium phosphate and phosphorylated serine. Indeed, tetracalcium phosphate undergoes a dissolution-precipitation reaction to hydroxyapatite spontaneously. By the addition of phosphorylated serine, this reaction is prevented and results in the formation of calcium phosphoserine monohydrate which forms a coordinated network allowing the adhesion to bone tissue and the initiation of bone calcification. Polydopamine, through its adhesive and osteogenic induction properties, potentiates the adhesive effect by promoting the bone reconstruction.

The same preparation was carried out this time without the addition of polydopamine nanoparticle powder. This preparation is a comparative composition such as can be found in the prior art.

### Example 2: Mechanical Tensile Tests on Titanium and Bovine Bones of the Composition According to the Invention

Metal (titanium) and bone (bovine bones) samples were used. The metal samples consisting of titanium cylinders with a gluing surface of 200 mm². The bone samples consisted of paired rectangular parallelepipeds with a gluing surface of 100 to 180 mm².

The adhesive compositions prepared in Example 1 or a suspension of polydopamine nanoparticles as obtained in Example 1, were applied in a thin layer to the surfaces of the samples (N=8). A manual compression of the samples therebetween was carried out and maintained for 4 minutes. The glued samples were then immersed for 1 hour or 24 hours in a PBS bath at 37° C. to simulate the physiological aqueous environment.

Then, the mechanical evaluation of the adhesion force was carried out in tensile (end-to-end formation) until rupture on an Instron 4466 machine (Norwood, MA, United States). The load cell was 1000 N when testing with the bone samples and 10,000 N when testing with the titanium samples. The movement speed was 0.1 mm/s.

The results were described in the form of tensile stress, i.e. the breaking force per unit area applied to the glued samples. The results were statistically analysed non-parametrically using the Mann-Whitney test.

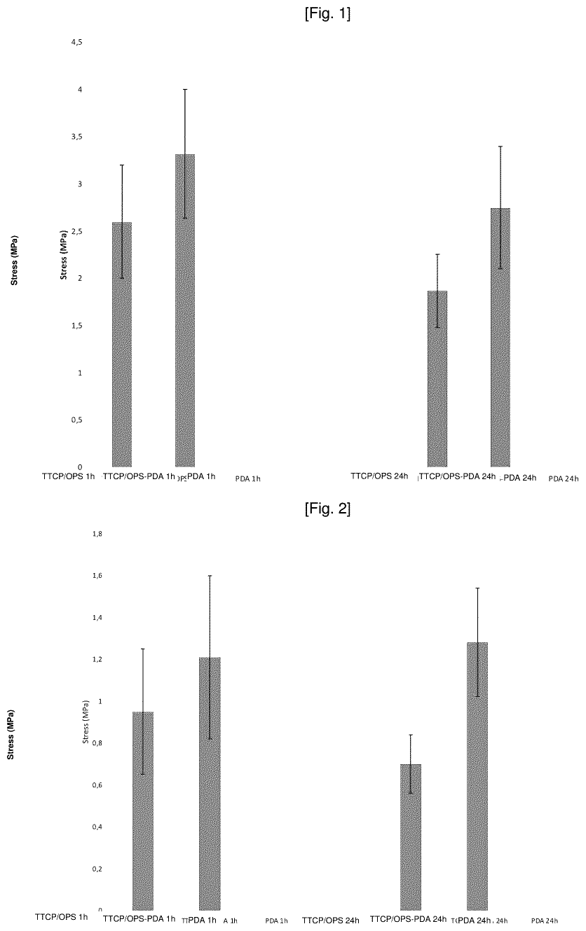

The results for the titanium samples (see FIG. 1) demonstrate that the adhesion was significantly higher with the composition according to the invention (TTCP/OPS-nPDA) relative to the comparative composition (TTCP/OPS) and to the suspension of polydopamine nanoparticles at 24 hours (p=0.0029).

The results for the bovine bone samples (see FIG. 2) demonstrate that the adhesion was significantly higher with the composition according to the invention (TTCP/OPS-nPDA) relative to the comparative composition (TTCP/OPS) and to the suspension of polydopamine nanoparticles at 24 hours (p=0.00093).

### Example 3: Ex Vivo Tensile Tests of the Composition According to the Invention

The following test was designed to be closer to a clinical situation by imitating the performance of a bone autograft. Samples of tibias and fibula fragments (6 mm long by 1 mm wide) from freshly sacrificed rats were used. The tibia was slightly sharpened on the outer face thereof, similarly to what is made before the placement of a bone autograft.

The adhesive compositions prepared in Example 1, with and without nPDA, were applied in a thin layer to the tibia (N=7). A Vicryl thread was applied then the fibula fragment was placed. A compression of the samples therebetween was carried out and maintained for 4 minutes. The glued samples were then immersed for 1 hour or 24 hours in a PBS bath at 37° C. to simulate the physiological aqueous environment.

Then, the mechanical evaluation of the adhesion force was carried out tensilely by placing standardised weights of increasing mass on the Vicryl thread until rupture (FIG. 2).

The results were described in the form of tensile stress, i.e. the breaking force per unit area applied to the glued samples. The results were statistically analysed non-parametrically using the Mann-Whitney test.

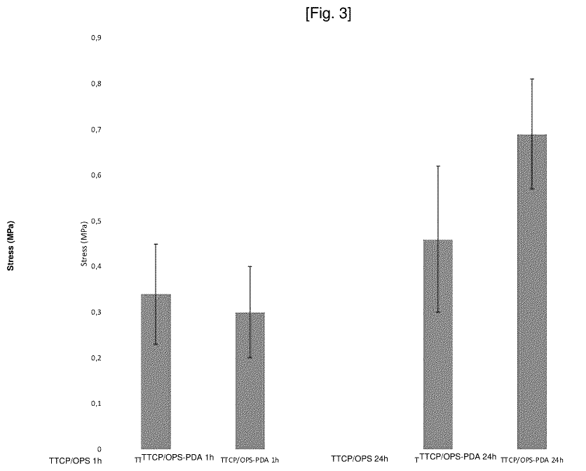

The results on the samples of tibias and fibula fragments (see FIG. 3) demonstrate that the adhesion was significantly higher with the composition according to the invention (TTCP/OPS-nPDA) relative to the comparative composition (TTCP/OPS) at 24 hours (p=0.029).

1-12. (canceled)

13. An adhesive composition comprising:
a calcium phosphate ceramic selected from tetracalcium phosphate and alpha-tricalcium phosphate, preferably tetracalcium phosphate,
phosphorylated serine,
polydopamine, and
an aqueous solvent.

14. The composition according to claim 13, wherein the amount of calcium phosphate ceramic is comprised between 50% and 80%, preferably between 60% and 70%, more preferably between 65 and 68% by dry weight.

15. The composition according to claim 13, wherein the amount of phosphorylated serine is comprised between 20% and 50%, preferably between 30% and 40%, more preferably between 32% and 35% by dry weight.

16. The composition according to claim 13, wherein the calcium phosphate ceramic/phosphorylated serine dry weight ratio is comprised between 1.5 and 2.5, preferably between 1.7 and 2.3, more preferably between 1.9 and 2.

17. The composition according to claim 13, wherein the amount of polydopamine is comprised between 1% and 5%, preferably 2% by dry weight.

18. The composition according to claim 13, wherein the density of the composition is comprised between 2 g/cm3 and 2.2 g/cm3.

19. The composition according to claim 13, wherein the polydopamine is functionalised with at least one active ingredient selected from antibiotics, osteoinductive molecules and x-ray contrast agents.

20. A kit for preparing an adhesive composition according to claim 13, comprising:
a calcium phosphate ceramic selected from tetracalcium phosphate and alpha-tricalcium phosphate, preferably tetracalcium phosphate,
phosphorylated serine, and
polydopamine.

21. The kit according to claim 20, wherein the calcium phosphate ceramic, the phosphorylated serine and the polydopamine are in powder form, preferably the polydopamine is in the form of nanoparticle powder.

22. A method for preparing an adhesive composition according to claim 13, comprising the following steps:
a) mixing a calcium phosphate ceramic selected from tetracalcium phosphate and alpha-tricalcium phosphate, preferably tetracalcium phosphate, phosphorylated serine and polydopamine in a container,
b) adding a solvent to the previous preparation,
c) recovering the mixture thus formed.

23. The method according to claim 22, wherein the calcium phosphate ceramic, the phosphorylated serine and the polydopamine are in powder form, preferably the polydopamine is in the form of nanoparticle powder.

24. The adhesive composition according to claim 13, for its therapeutic use in vivo as a bone adhesive.

25. A method of treating a bone of a subject in need thereof, comprising applying to the bone of the subject the adhesive composition according to claim 13.
